(19) United States
(12) Patent Application Publication
(10) Pub. No.: US 2024/0148376 A1
(43) Pub. Date: May 9, 2024

(54) SMALL DIAMETER LINEAR SURGICAL STAPLING APPARATUS

(71) Applicant: Covidien LP, Mansfield, MA (US)

(72) Inventor: Justin P. Williams, Southbury, CT (US)

(21) Appl. No.: 18/413,627

(22) Filed: Jan. 16, 2024

Related U.S. Application Data
(63) Continuation of application No. 18/109,484, filed on Feb. 14, 2023, now Pat. No. 11,896,220.
Application No. 18/109,484 is a continuation of application No. 17/407,623, filed on Aug. 20, 2021, now Pat. No. 11,576,671.

(51) Int. Cl. A61B 17/068; A61B 17/072

(52) U.S. Cl. CPC A61B 17/0686; A61B 17/072; A61B 2017/00367

(57) ABSTRACT

A surgical stapling apparatus includes a housing assembly and an adapter assembly defining a longitudinal axis and selectively attachable to the housing assembly. The adapter assembly supports an end effector including an anvil assembly, a cartridge assembly, and a spring lock. The cartridge assembly supports a plurality of fasteners and a sled that is movable through the cartridge assembly to fire the plurality of fasteners. The spring lock is supported on the sled. The spring lock is positioned to move from an unlocked position to a locked position when the sled moves through the cartridge assembly to prevent the cartridge assembly from being refired.

### CROSS-REFERENCE TO RELATED APPLICATIONS

This application is a continuation of U.S. patent application Ser. No. 18/109,484, filed Feb. 14, 2023, which is a continuation of U.S. patent application Ser. No. 17/407,623, filed on Aug. 20, 2021, now U.S. Pat. No. 11,576,671, the entire contents of each of which are incorporated by reference herein.

### TECHNICAL FIELD

This disclosure relates to surgical stapling apparatus and, more particularly, to surgical stapling apparatus configured to rotate, articulate, and fire within small diameter (e.g., 8 mm) passages.

### BACKGROUND

Fasteners have traditionally been used to replace suturing when joining various body structures. Surgical stapling apparatus employed to apply these fasteners are generally designed to simultaneously cut and seal tissue to reduce the time and risks involved with surgical procedures. Surgical stapling apparatus that clamp, cut and/or staple tissue are well known in the art. Such surgical stapling apparatus include end effectors having two elongated jaw members used to capture or clamp tissue. These end effectors can be provided in the form of an elongate loading unit removably attachable to a housing assembly via an adapter to enable drive components of the housing assembly to operate the end effector in vivo, for instance, laparoscopically. In particular, one of the two jaw members of the end effector usually carries a staple cartridge that houses a plurality of staples positioned in rows, while the other of the two jaw members has an anvil for forming the staples as the staples are driven from the staple cartridge. In linear surgical stapling apparatus, for example, a stapling operation is effectuated by a cam bar, a drive sled or other similar mechanism having a cam member that travels longitudinally through channels defined in the staple cartridge and acts upon staple pushers in the channels to sequentially eject linear rows of staples from the staple cartridge. A knife is movably positioned between the linear rows of staples such that when the surgical stapling apparatus is positioned about tissue and actuated, the tissue is joined and/or simultaneously or nearly simultaneously cut.

### SUMMARY

Surgical stapling apparatus are challenging to design, particularly when such instruments are required to fit rotation, articulation, and firing within small diameter tubes that are as small as 8 mm for providing tight access and/or effectuating thin and/or vascular tissue stapling. When cartridge detection technology is required, adding the supplementary challenge of incorporating electronic chip technology within the limited space provided by the small diameter, design efforts become further complicated.

According to one aspect, a surgical stapling apparatus includes a housing assembly and an adapter assembly. The adapter assembly defines a longitudinal axis and is selectively attachable to the housing assembly. The adapter assembly includes an end effector and an articulation assembly. The articulation assembly includes an articulation drive screw, an articulation nut threadedly mounted to the articulation drive screw, an articulation yolk coupled to the articulation nut, and an articulation tube that is positioned to move in response to movement of the articulation nut. Movement of the articulation tube causes the end effector to articulate relative to the longitudinal axis.

In aspects, the articulation tube may define a slot therein that receives the articulation yolk. The articulation tube may support an articulation joint on a distal end portion thereof. The articulation joint may be coupled to one or more articulation links that are axially movable to articulate the end effector in response to axial movement of the articulation tube. The articulation links may include a proximal articulation link and a distal articulation link. The distal articulation link may have a proximal end portion that is coupled to the proximal articulation link. The distal articulation link may have a distal end portion that is pivotably coupled to an articulation pin of the end effector.

In aspects, the surgical stapling apparatus may further include a firing assembly including a drive beam assembly that is positioned to advance through the end effector. The firing assembly may further include a firing drive screw that is positioned within the articulation tube. The firing drive screw may be threadedly coupled to a firing nut. The firing nut may be coupled to a firing tube. The firing tube may be axially advanceable through the articulation tube in response to axial movement of the firing nut. The firing nut may be positioned to translate in response to rotation of the firing drive screw. A firing rod may be secured to a distal end portion of the firing tube. The firing rod may be coupled to the drive beam assembly and positioned to advance the drive beam assembly through the end effector when the firing tube translates the firing rod distally.

In aspects, the surgical stapling apparatus may further include a rotation assembly having a driver that couples to a pinion. The pinion may be coupled to an annulus. The annulus may be coupled to a housing on a proximal end portion of the adapter assembly. The housing may be coupled to an outer tube. The outer tube may be coupled to the end effector, wherein rotation of the driver causes the end effector to rotate about the longitudinal axis.

According to one aspect, this disclosure is directed to a surgical stapling apparatus including a housing assembly, an elongated shaft assembly, an anvil assembly, a cartridge assembly, and an articulation assembly. The elongated shaft assembly defines a longitudinal axis and has a distal end portion and a proximal end portion. The proximal end portion is selectively attachable to the housing assembly. The anvil assembly is supported on the distal end portion of the elongated shaft assembly. The cartridge assembly is selectively attachable to the anvil assembly to secure the cartridge assembly to the elongated shaft assembly. The articulation assembly includes an articulation drive screw, an articulation nut threadedly mounted to the articulation drive screw, an articulation yolk coupled to the articulation nut, and an articulation tube that is positioned to move in response to movement of the articulation nut, wherein movement of the articulation tube causes the anvil and cartridge assemblies to articulate relative to the longitudinal axis.

In aspects, the anvil assembly may include an anvil and tissue stops on a proximal end portion thereof. The tissue stops may define cartridge tab slots therethrough, and wherein the cartridge assembly may include proximal tabs that are receivable within the cartridge tab slots of the anvil assembly to couple the cartridge assembly to the anvil assembly. The cartridge assembly may include a cartridge and a support plate. The proximal tabs may extend from the support plate.

In aspects, the articulation tube may define a slot therein that receives the articulation yolk. The articulation tube may support an articulation joint on a distal end portion thereof. The articulation joint may be coupled to one or more articulation links that are movable to articulate the anvil and cartridge assemblies in response to movement of the articulation tube. The one or more articulation links may include a proximal articulation link and a distal articulation link. The distal articulation link may have a proximal end portion that is coupled to the proximal articulation link. The distal articulation link may have a distal end portion that is pivotably coupled to an articulation pin of the anvil assembly.

According to yet another aspect, this disclosure is directed to an adapter assembly of a surgical stapling apparatus. The adapter assembly includes a proximal housing, an articulation assembly, an elongated shaft assembly, and an end effector. The articulation assembly is supported by the proximal housing. The articulation assembly includes an articulation drive screw, an articulation nut threadedly mounted to the articulation drive screw, an articulation yolk coupled to the articulation nut, and an articulation tube positioned to move in response to movement of the articulation nut. The elongated shaft assembly is coupled to the proximal housing and defines a longitudinal axis. The elongated shaft assembly has a proximal end portion and a distal end portion. The end effector is supported on the distal end portion of the elongated shaft assembly and includes an anvil assembly and a cartridge assembly selectively attachable to the anvil assembly. Rotation of the articulation drive screw causes the end effector to articulate relative to the longitudinal axis.

In aspects, the adapter assembly may further include a firing assembly. The firing assembly may include a drive beam assembly that is positioned to advance through the cartridge assembly. The firing assembly may further include a firing drive screw that is positioned within the articulation tube. The firing drive screw may be threadedly coupled to a firing nut. The firing nut may be coupled to a firing tube. The firing tube may be being axially advanceable through the articulation tube in response to axial movement of the firing nut. The firing nut may be positioned to translate in response to rotation of the firing drive screw.

In aspects, a firing rod may be secured to a distal end portion of the firing tube. The firing rod may be coupled to the drive beam assembly and positioned to advance the drive beam assembly through the end effector when the firing tube translates the firing rod distally.

Other aspects, features, and advantages will be apparent from the description, the drawings, and the claims that follow.

### BRIEF DESCRIPTION OF DRAWINGS

The accompanying drawings, which are incorporated in and constitute a part of this specification, illustrate aspects of the disclosure and, together with a general description of the disclosure given above and the detailed description given below, explain the principles of this disclosure, wherein:

FIG. 1 is a perspective view of an exemplary surgical stapling apparatus in accordance with the principles of this disclosure;

FIG. 2 is a perspective view of the surgical stapling apparatus of FIG. 1 with an elongated shaft assembly of the surgical stapling apparatus shown separate from a housing assembly of the surgical stapling apparatus, the elongated shaft assembly including an end effector supported on a distal end portion thereof;

FIG. 3 is an enlarged view of the indicated area of detail shown in FIG. 2, the view illustrating the end effector;

FIG. 4 is a perspective view illustrating a cartridge assembly of the end effector of FIG. 3 separated from an anvil assembly of the end effector of FIG. 3;

FIG. 5 is a perspective view of the anvil assembly of FIG. 4;

FIG. 6 is a perspective view, with parts separated, of the cartridge assembly of FIG. 4;

FIG. 7 is an enlarged view of a sled and a lockout spring of the cartridge assembly of FIG. 6;

FIG. 8 is an enlarged perspective view of a proximal end portion of the elongated shaft assembly with portions removed for clarity;

FIG. 9 is a perspective view, with parts separated, of the elongated shaft assembly;

FIG. 10 is a perspective view of the elongated shaft assembly with portions thereof removed for clarity;

FIG. 11 is an enlarged perspective view of the indicated area of detail shown in FIG. 9;

FIG. 12 is an enlarged perspective view of the indicated area of detail shown in FIG. 10;

FIG. 13 is a perspective view of a distal portion of the elongated shaft assembly with portions thereof shown in phantom for clarity;

FIGS. 14 and 15 are perspective views of the elongated shaft assembly with portions thereof removed or shown in phantom for clarity;

FIG. 16 is an enlarged view of the indicated area of detail shown in FIG. 15;

FIG. 17 is a cross-sectional view of the elongated shaft assembly as taken along section line 17-17 shown in FIG. 2;

FIGS. 18 and 19 are enlarged views of the indicated areas of detail shown in FIG. 17;

FIG. 20 is a cross-sectional view of the elongated shaft assembly as taken along section line 20-20 shown in FIG. 2;

FIG. 21 is a cross-sectional view as taken along section line 21-21 shown in FIG. 20;

FIG. 22 is a cross-sectional view as taken along section line 22-22 shown in FIG. 21;

FIGS. 23 and 24 are progressive views illustrating articulation of the end effector relative to a longitudinal axis of the elongated shaft assembly;

FIG. 25 is a cross-sectional view of a proximal portion of the elongated shaft assembly with portions thereof shown in phantom for clarity;

FIG. 26 is a perspective, cross-sectional view of the elongated shaft assembly as taken along section line 26-26 shown in FIG. 10;

FIG. 27 is an enlarged view of the indicated area of detail shown in FIG. 26; and

FIG. 28 is an enlarged view of a distal portion of FIG. 26 illustrating the lockout spring of FIG. 7 in a locked position.

### DETAILED DESCRIPTION

Aspects of the disclosed surgical stapling apparatus are described in detail with reference to the drawings, in which like reference numerals designate identical or corresponding elements in each of the several views. As commonly known, the term “clinician” refers to a doctor, a nurse, or any other care provider and may include support personnel. Additionally, the term “proximal” refers to the portion of structure that is closer to the clinician and the term “distal” refers to the portion of structure that is farther from the clinician. In addition, directional terms such as front, rear, upper, lower, top, bottom, and the like are used simply for convenience of description and are not intended to limit the disclosure attached hereto.

In the following description, well-known functions or constructions are not described in detail to avoid obscuring the present disclosure in unnecessary detail.

Further, although the surgical instrument described herein is provided in connection with a powered laparoscopic surgical stapling apparatus for brevity, the disclosed surgical instrument can include any powered, manual, or robotically-controlled surgical instruments such as a clip applier, stitching device, energy-based device (e.g., a bipolar or monopolar forceps) or the like, and/or other surgical stapling apparatus such as a circular stapler, a transverse stapler, or an open stapler. For a detailed description of the structure and function of exemplary surgical stapling apparatus, one or more components of which may be included, or modified for use with the disclosed aspects, reference may be made to U.S. Pat. Nos. 9,713,470; 8,806,973; 8,256,656; 8,157,152; 8,070,033 7,819,896; 7,770,774; 7,334,717; 7,128,253; 5,964,394; and 5,915,616, the entire contents of each of which are incorporated herein by reference.

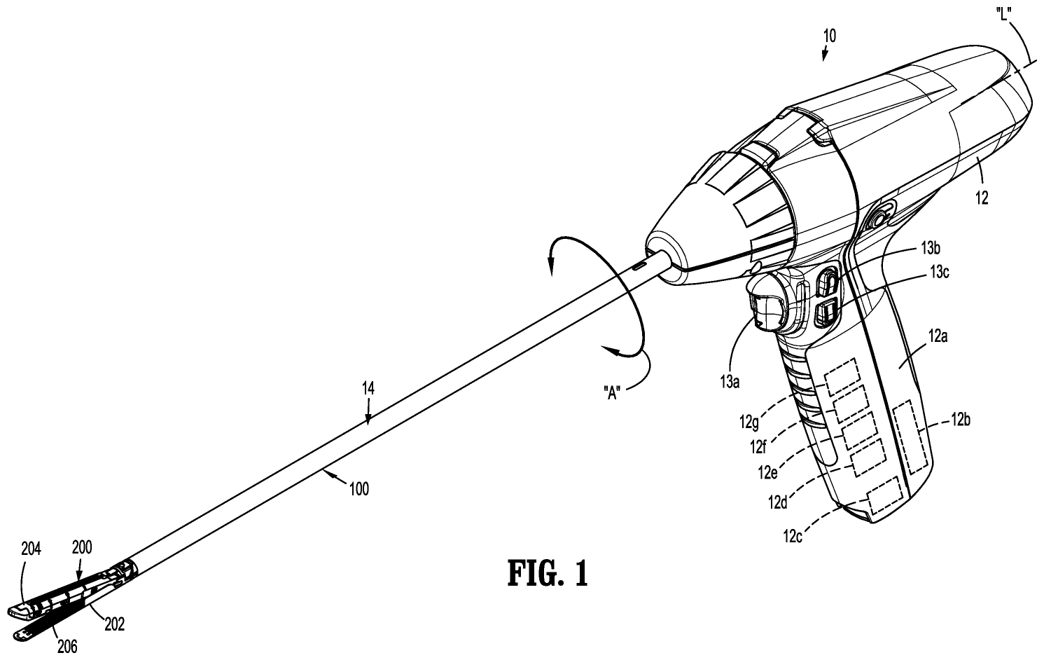

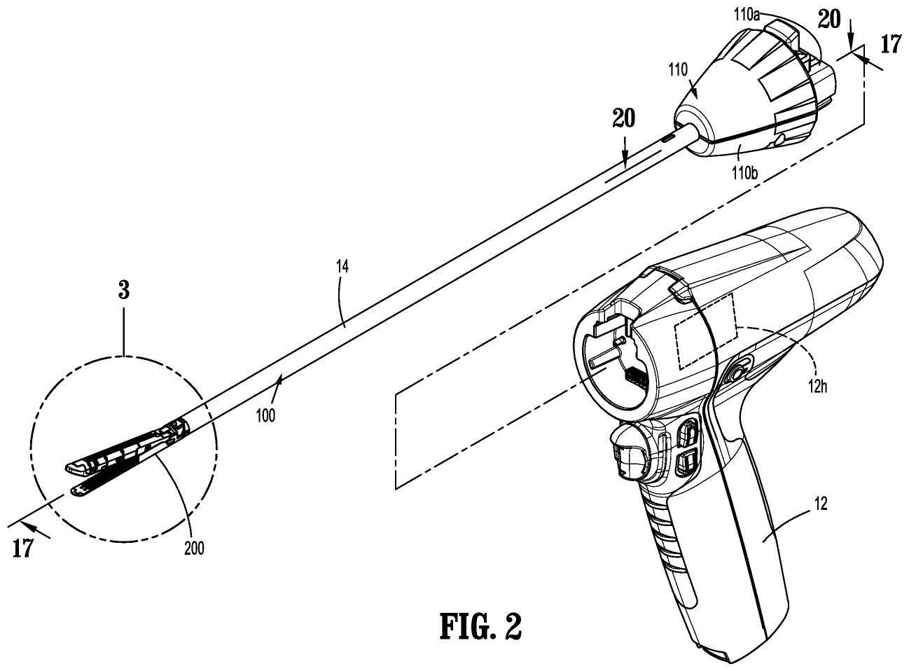

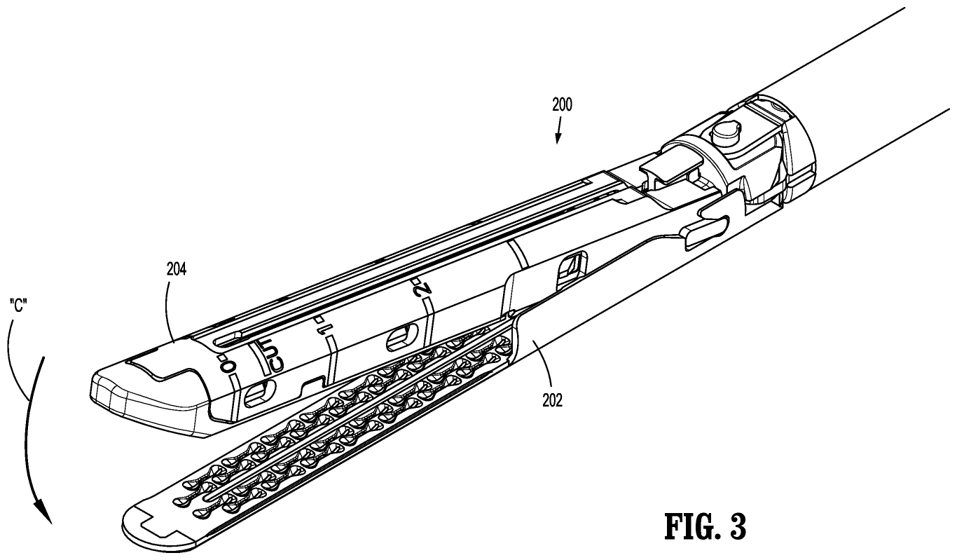

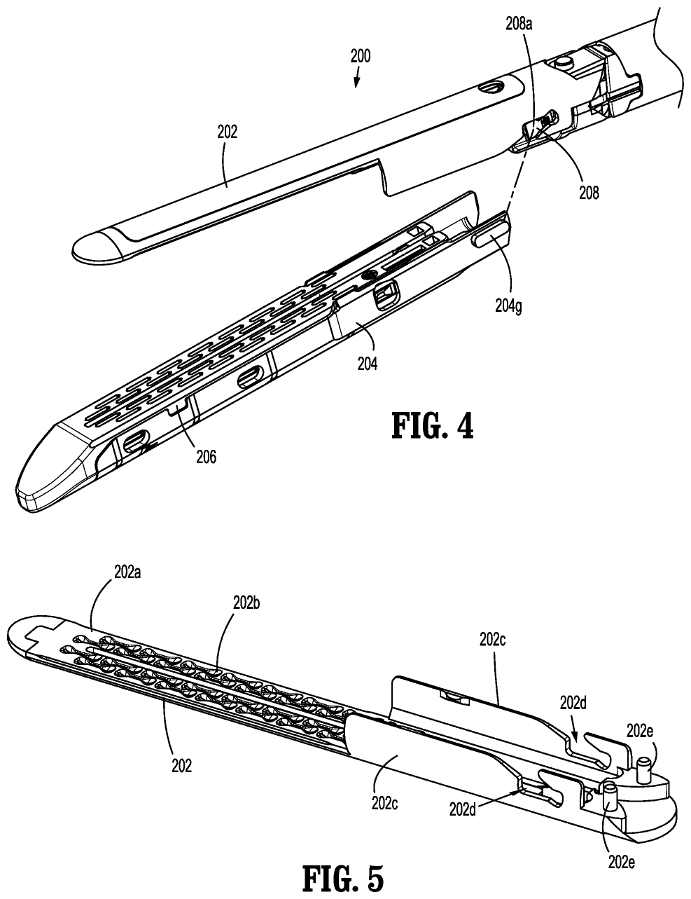

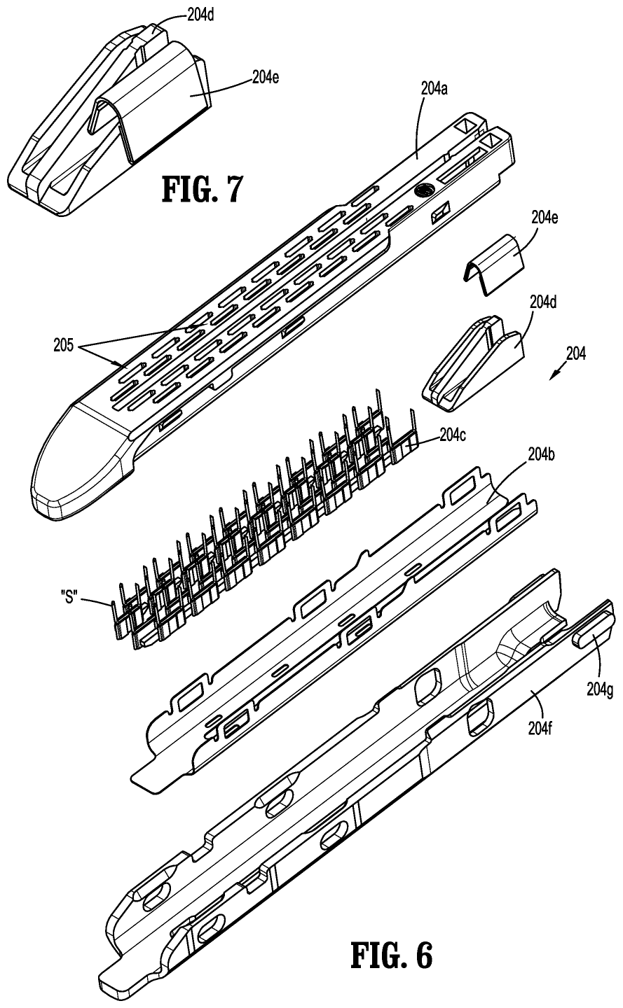

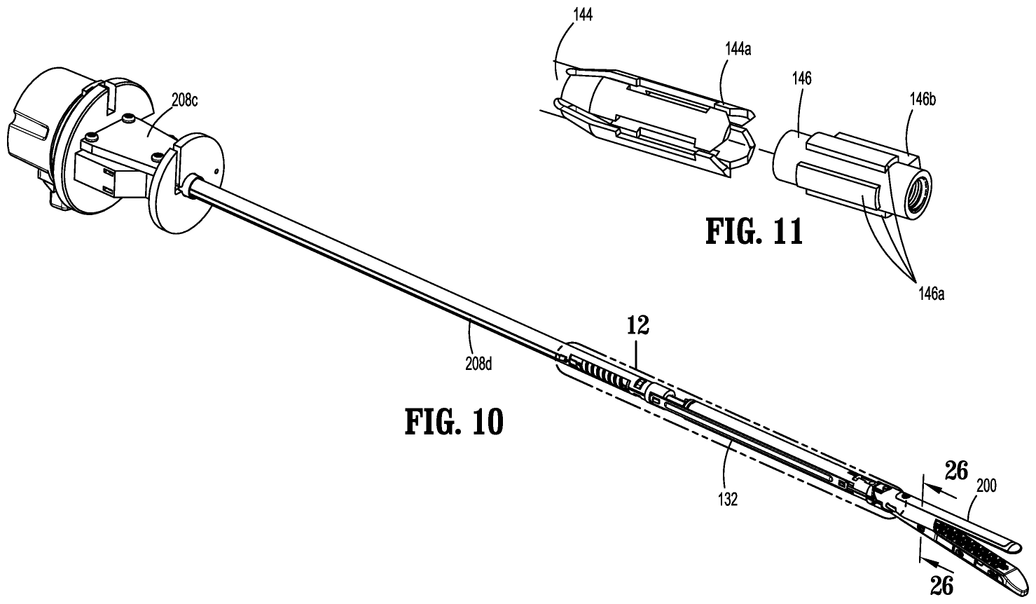

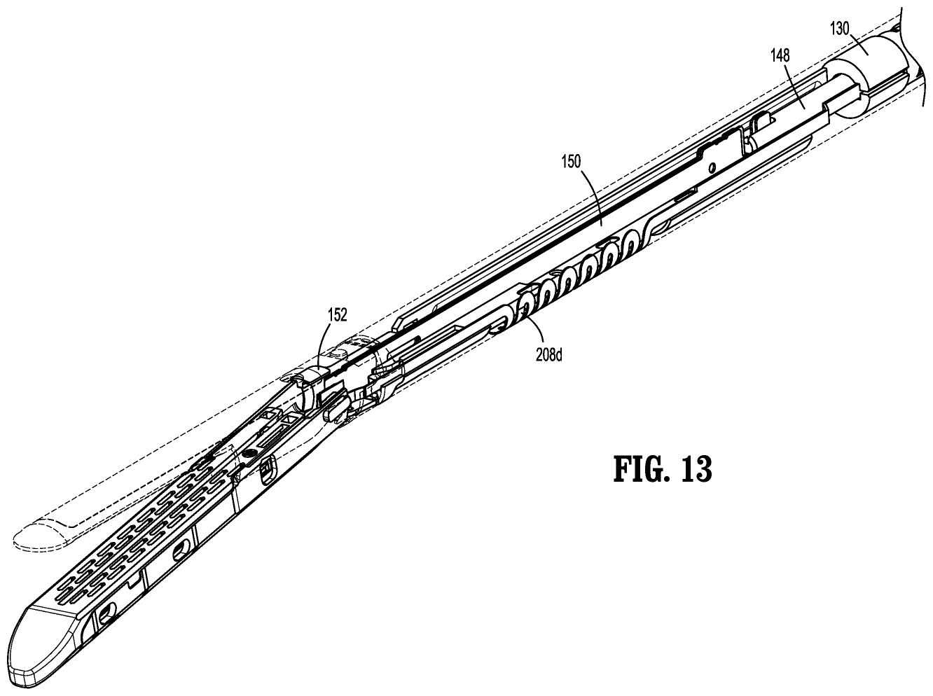

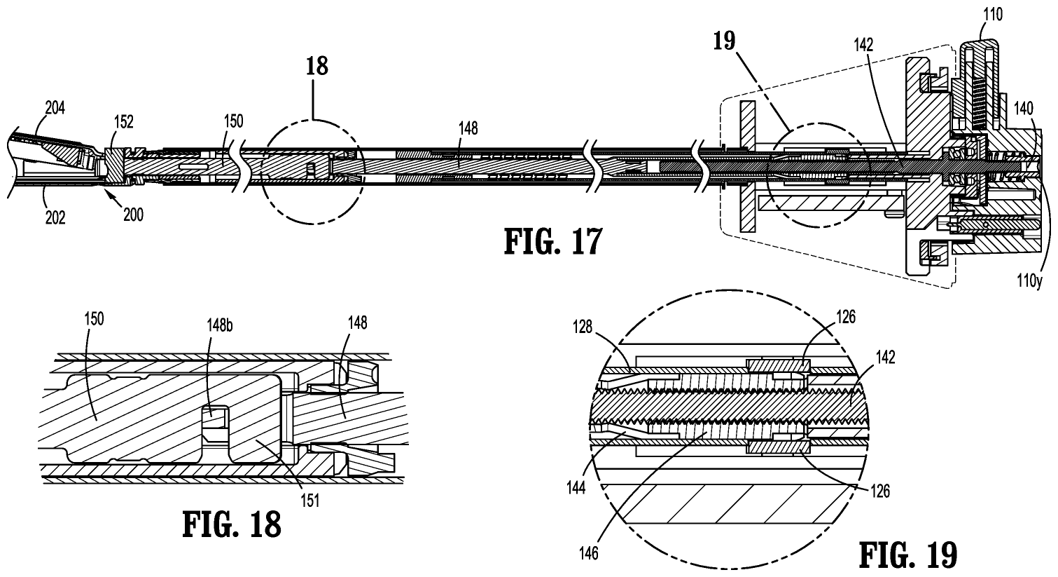

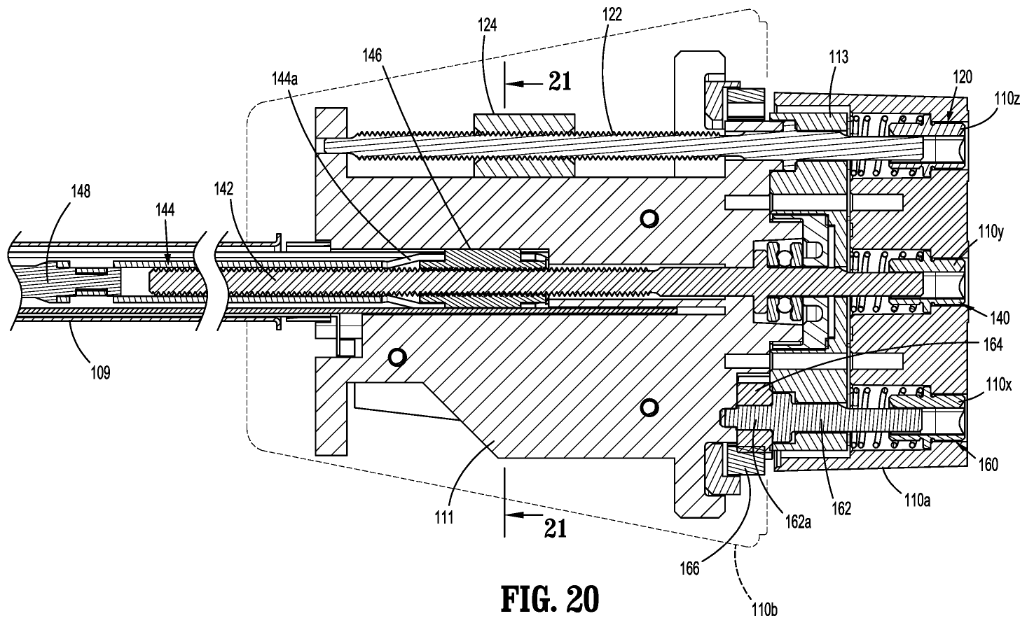

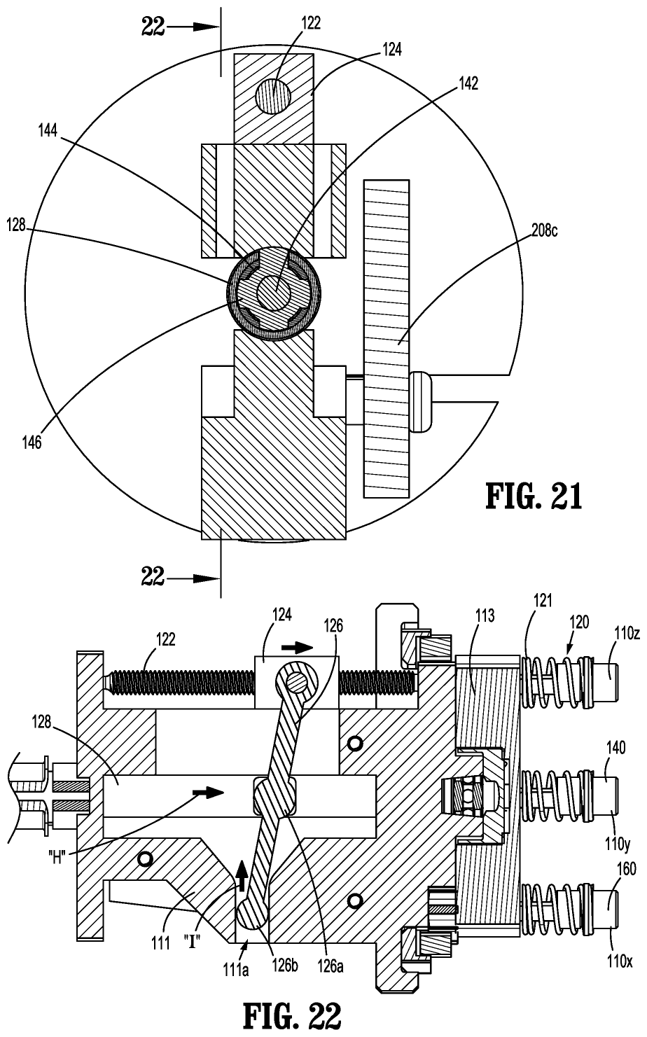

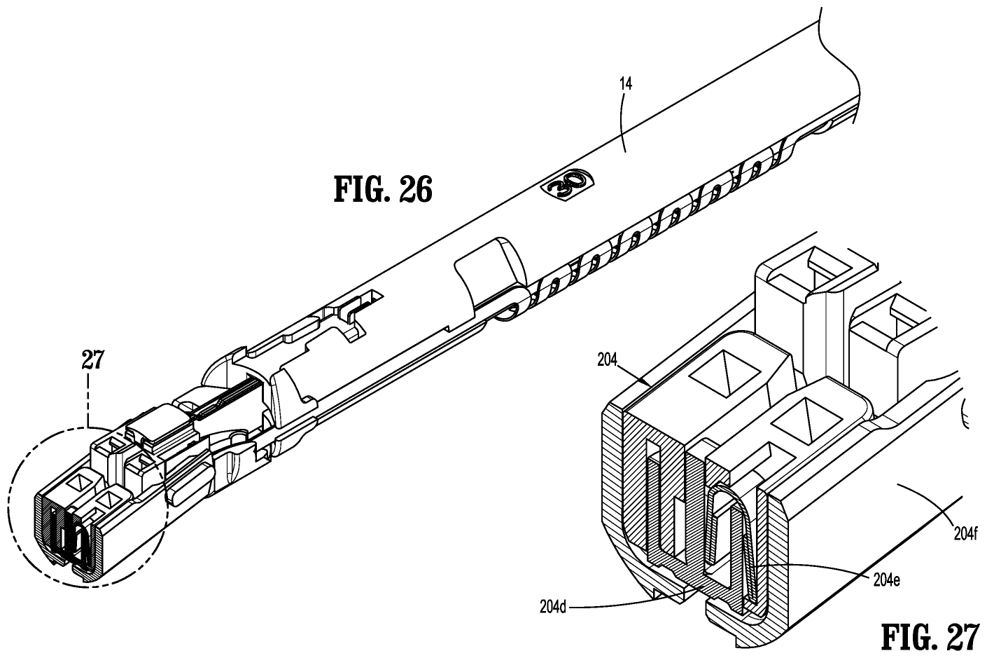

Turning now to FIGS. 1-28, this disclosure is directed to a surgical stapling apparatus 10. With reference to FIGS. 1-7, surgical stapling apparatus 10 includes a housing assembly 12 (which may include one or more handles that may be manually actuatable to fire surgical stapling apparatus 10) and an elongated shaft assembly 14 that is removably secured to housing assembly 12. Elongated shaft assembly 14 extends distally from housing assembly 12 and defines a longitudinal axis “L” therealong. Elongated shaft assembly 14 includes an adapter assembly 100 having a proximal end portion removably secured to housing assembly 12. Adapter assembly 100 extends distally from housing assembly 12 to an end effector 200. End effector 200 includes an anvil assembly 202 and a cartridge assembly 204 that houses a plurality of fasteners (e.g., staples “S”) (FIG. 6) in a reload or cartridge 206 thereof that may be reusable and/or disposable and selectively replaceable.

Anvil assembly 202 of end effector 200 includes an anvil 202a defining tissue pockets 202b against which the plurality of staples “S” is formed upon a firing of surgical stapling apparatus 10. Anvil assembly 202 further includes tissue stops 202c on a proximal end portion thereof. The tissue stops 202c define cartridge tab slots 202d therethrough configured to secure cartridge assembly 204 to anvil assembly 202. Anvil assembly 202 further includes articulation posts 202e on a proximal end portion thereof for facilitating pivotal (articulating) movement of end effector 200 relative to longitudinal axis “L.”

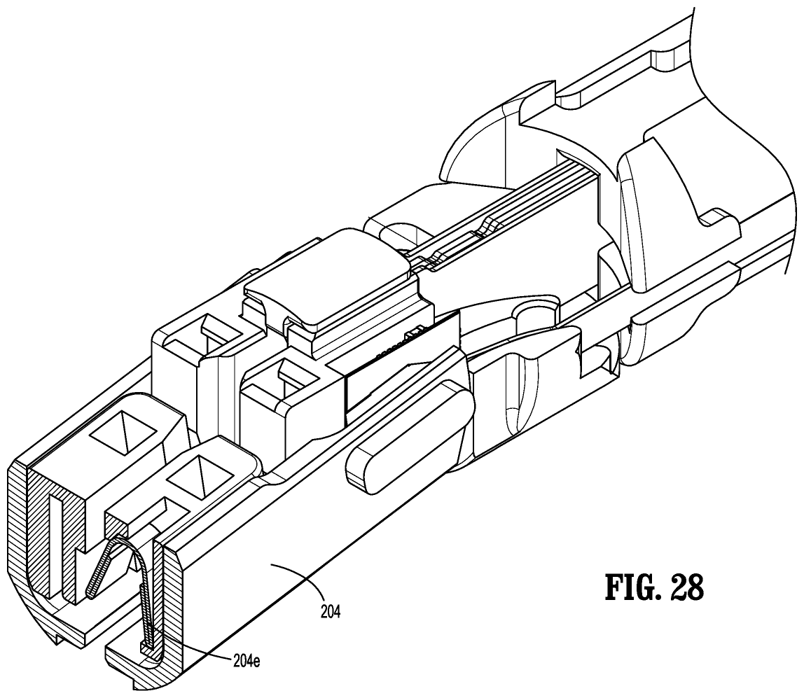

Cartridge assembly 204 of end effector 200 includes an upper cartridge body 204a and a lower cartridge body 204b that support fasteners, pushers 204c, and a sled 204d therebetween. Upper cartridge body 204a defines a plurality of staple slots 205 that supports fasteners therein. A spring lock 204e is supported on sled 204d and positioned to spring open from an unlocked position (FIG. 27) to a locked position (FIG. 28) when sled 204d advances distally through cartridge assembly 204 to prevent cartridge assembly 204 from being refired. Cartridge assembly 204 further includes a support plate 204f that couples to upper and lower cartridge bodies 204a, 204b. Support plate 204f includes proximal tabs 204g on opposite sides thereof that are received within cartridge tab slots 202d of anvil assembly 202 to secure cartridge assembly 204 to anvil assembly 202.

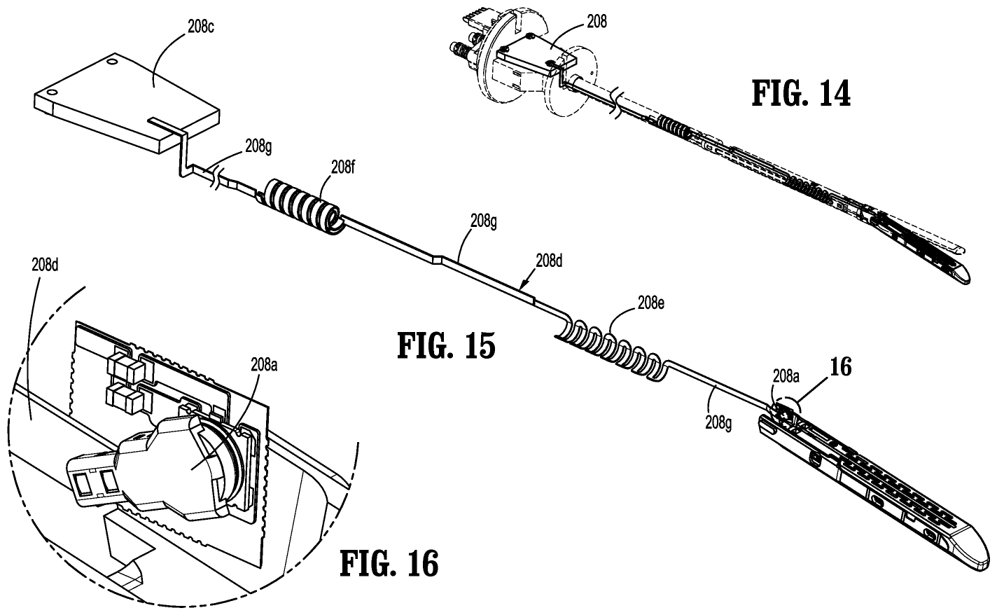

End effector 200 further includes a sensor assembly 208 supported by anvil assembly 202. Sensor assembly 208 may include a cartridge ID sensor or cartridge detector 208a disposed in electrical communication with housing assembly 12 (e.g., a controller thereof). The cartridge detector 208a is disposed in registration with cartridge tab slots 202d such that cartridge detector 208a is configured to detect when proximal tabs 204g of cartridge assembly 204 are received within cartridge tab slots 202d of anvil assembly 202 and communicate a signal to a controller 208c of sensory assembly 208 via a flex cable 208d extending through elongated shaft assembly 14 (see FIGS. 14-16). The signal indicative of the securement of the cartridge assembly 204 to the anvil assembly 202. The controller 208c disposed within a proximal end portion of elongated shaft assembly 14. Flex cable 208d includes a distal linear undulation 208e and a proximal rotational coil 208f that are coupled together, and to controller 208c and cartridge detector 208a, via various cable segments 208g. Controller 208c is configured to electrically communicate with housing assembly 12 (e.g., with a controller 12c supported within housing assembly 12).

Housing assembly 12 of surgical stapling apparatus 10 includes a housing 12a configured for selective removable receipt of a rechargeable battery 12b. Battery 12b is configured to supply power to electrical components of surgical stapling apparatus 10. Housing 12a supports controller 12c (e.g., a circuit board) therein. Controller 12c is configured to control various operations of surgical stapling apparatus 10 and includes any number of electronic components such as a memory 12d, a processor 12e, a network interface 12f, and/or other input/output modules 12g. Controller 12c may be coupled to a local or remote display device (not shown) for outputting information and/or data such as a condition of components of surgical stapling apparatus 10 and/or tissue grasped by end effector 200.

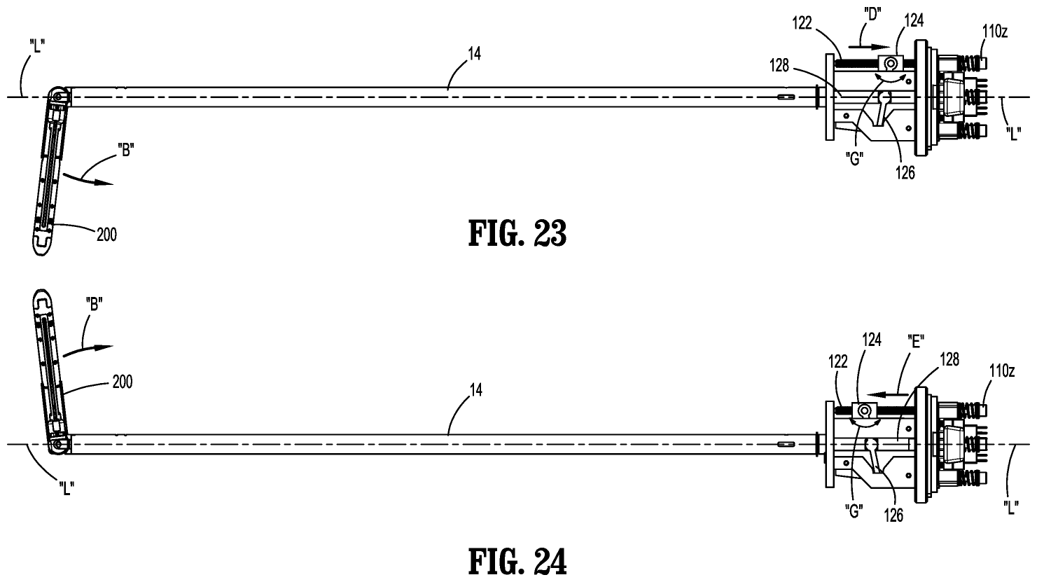

Surgical stapling apparatus 10 further includes a drive mechanism 12h configured to drive mechanical and/or electrical components such as rotatable shafts and/or gear components (not shown) within housing 12a in order to perform various operations of surgical stapling apparatus 10. For instance, drive mechanism 12h may be operable to selectively rotate and/or articulate end effector 200 about, and/or relative to, the longitudinal axis “L” of surgical stapling apparatus 10, as indicated by arrows “A” (FIG. 1) and “B” (FIGS. 23 and 24), respectively; to selectively move cartridge assembly 204 relative to the anvil assembly 202 and/or vice versa, as indicated by arrow “C” to selectively clamp tissue; and/or to fire surgical stapling apparatus 10 for fastening and/or cutting the clamped tissue. Battery 12b, controller 12c, and/or drive mechanism 12h may be operably coupled to one or more actuators 13a, 13b, and 13c such as finger-actuated control buttons, rocker devices, and/or the like to effectuate various functions of surgical stapling apparatus 10 such as those described above.

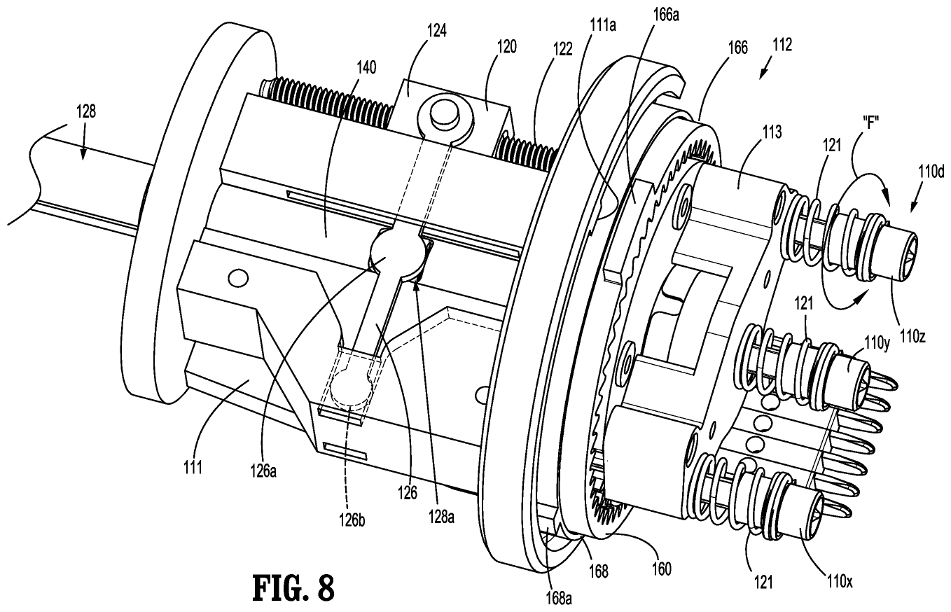

As best seen in FIGS. 8-28, adapter assembly 100 of elongated shaft assembly 14 includes an outer housing 110 on a proximal end portion thereof and an outer tube 109 that extends distally from outer housing 110 to a support assembly 108. Support assembly 108 includes an upper shell 108a and a lower shell 108b that couple together to support a drive beam assembly 150 therein. The support assembly 108 has a distal end portion pivotably coupled to a proximal end of end effector 200 to enable end effector 200 to articulate relative to longitudinal axis “L.” Outer housing 110 supports a drive assembly 112 therein. Outer housing 110 has a proximal outer housing 110a and a distal outer housing 110b that extends distally from proximal outer housing 110a. Proximal outer housing 110a supports an electrical assembly 110c and a plurality of drive couplers 110d that electromechanically couple to drive mechanism 12h of housing assembly 12 for effecting rotation, articulation, and firing of surgical stapling apparatus 10. Electrical assembly 110c is configured to electrically communicate with, for example, controller 12c of housing assembly 12 when adapter 100 is coupled to housing assembly 12 and when drive couplers 110d mechanically engage drive mechanism 12h, which may include, for instance, a plurality of rotatable actuators (shown) to impart mechanical force (e.g., rotational force) through drive assembly 112 of adapter assembly 100. Specifically, a first actuator 110z is configured to facilitate articulation of end effector 200 relative to longitudinal axis “L,” a second actuator 110y is configured to fire the surgical stapling apparatus 10 for clamping, cutting, and fastening tissue, and a third actuator 110x is configured to rotate adapter assembly 100 (and thus end effector 200) about longitudinal axis “L” and relative to housing assembly 12. Actuators 110z, 110y, 110x are spring biased by springs 121 relative adapter assembly 100. Distal outer housing 110b includes an upper housing 110e and a lower housing 110f that define tab recesses 110g therein.

A proximal end portion of adapter assembly 100 further includes an inner body assembly 111 and an actuator mount 113 coupled to inner body assembly 111. The inner body assembly 111 and the actuator mount 113 are supported within outer housing 110.

Adapter assembly 100 of elongated shaft assembly 14 includes an articulation assembly 120 configured to articulate end effector 200 relative to longitudinal axis “L,” a firing assembly 140 configured to fire end effector 200 (e.g., to clamp, fasten, and cut tissue), and a rotation assembly 160 configured to rotate end effector 200 about longitudinal axis “L.”

Articulation assembly 120 of adapter assembly 100 includes first actuator 110z on a proximal end portion thereof that mechanically engages with drive mechanism 12h of housing assembly 12. Articulation assembly 120 also includes an articulation drive screw 122 that is mechanically coupled to first actuator 110z. Articulation drive screw 122 extends distally from first actuator 110z and through actuator mount 113. Articulation assembly 120 further includes an articulation nut 124 threadedly coupled to articulation drive screw 122 and axially advanceable therealong. Articulation nut 124 is configured to translate in proximal and/or distal directions, as indicated by arrows “D” and “E” (FIGS. 23 and 24), respectively, in response to rotation of articulation drive screw 122, as indicted by arrows “F” (FIG. 8). Articulation assembly 120 further includes a pair of articulation yolks 126 pinned to articulation nut 124 via pins 124a, 124b extending from articulation nut 124. Pins 124a, 124b enable articulation yolks 126 to pivot about pins 124a, 124b, as indicated by arrows “G” (FIGS. 23 and 24), when articulation nut 124 axially translates along articulation drive screw 122. Articulation yolks 126 include a drive plate 126a and an end plate 126b. Articulation assembly 120 also includes an articulation tube 128 supported within outer tube 109 and coupled to articulation yolks 126 via drive plates 126a. Drive plates 126a of articulation yolks 126 are received within drive notches 128a defined within a proximal end portion of articulation tube 128. End plates 126b of articulation yolks 126 are slidably (vertically as indicated by arrows “I” in FIG. 22) and rotatably received within a tapered guide channel 111a defined within inner body assembly 111. Drive plates 126a are configured to cause articulation tube 128 to axially translate, as indicated by arrows “H” (FIG. 22), as articulation yolks 126 pivot about articulation nut 124.

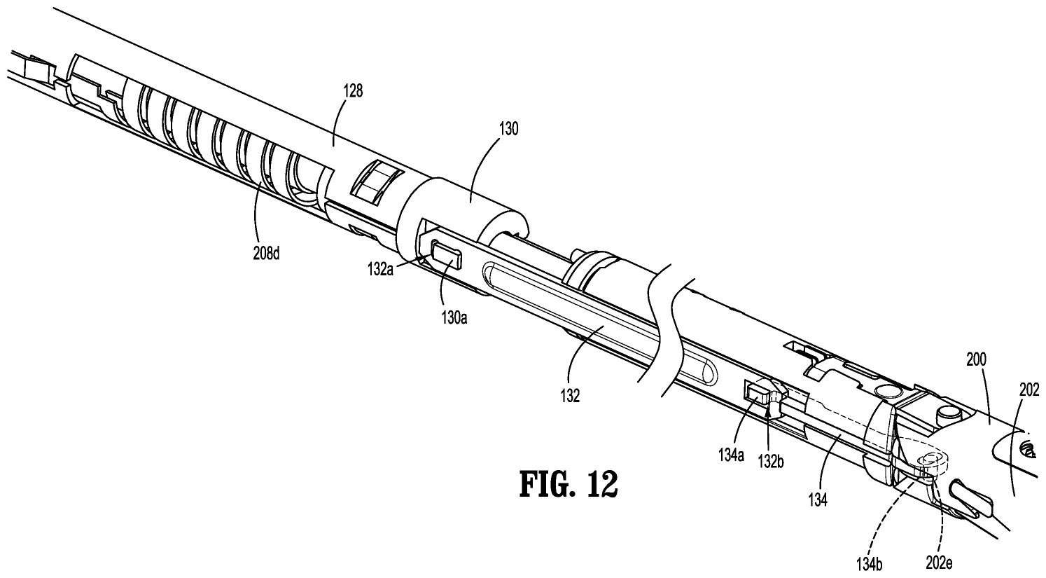

Articulation assembly 120 of adapter assembly 100 further includes an articulation joint 130 coupled to a distal end portion of articulation tube 128, proximal articulation links 132 that have proximal notches 132a coupled to distal tabs 130a on opposed sides of articulation joint 130, and distal articulation links 134 that are coupled to distal notches 132b of proximal articulation links 132. Distal articulation links 134 include proximal hooks 134a received within distal notches 132b of proximal articulation links 132 and distal apertures 134b (FIG. 12) defined therethrough that receive articulation posts 202e of anvil assembly 202 (see FIG. 5). Articulation joint 130 is positioned to provide a wire path channel therethrough for flex cable 208d.

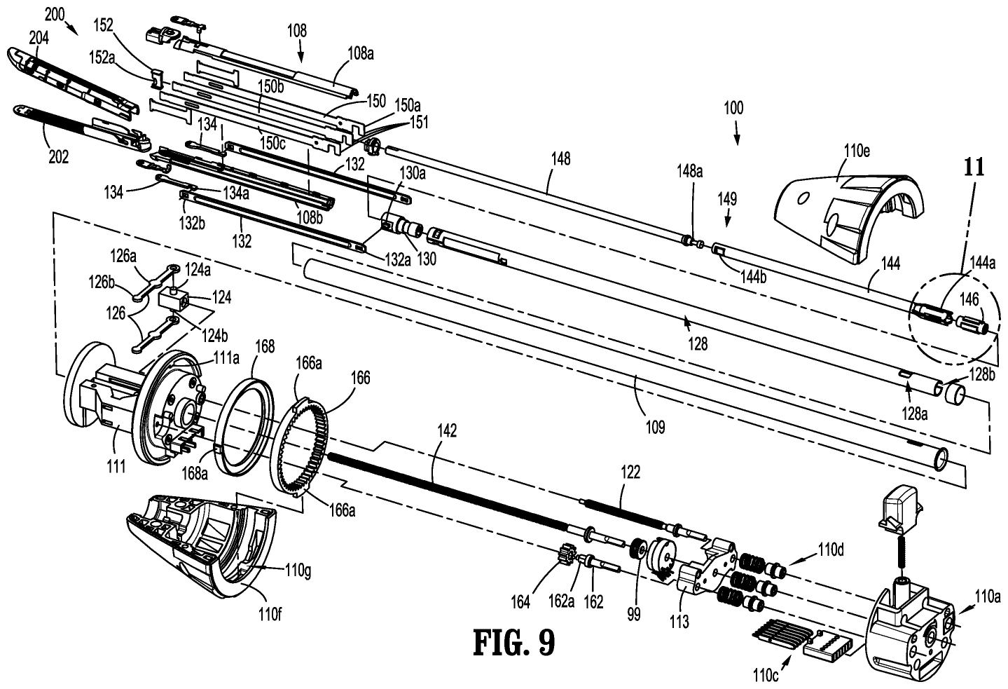

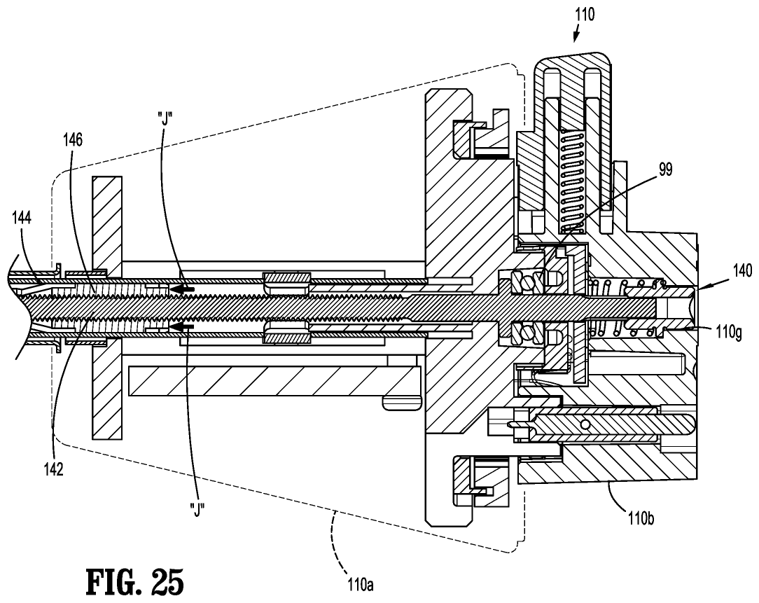

Firing assembly 140 of adapter assembly 100 includes second actuator 110y on a proximal end portion thereof that mechanically engages with drive mechanism 12h of housing assembly 12. Firing assembly 140 further includes a firing drive screw 142 that is mechanically coupled to second actuator 110y and extends distally therefrom and through inner body assembly 111. Firing drive screw 142 is rotatably supported within inner housing assembly 111 by a bearing assembly 99. A distal portion of firing drive screw 142 is coupled to a proximal end portion of a firing tube 144 via a firing nut 146 threadedly mounted on firing drive screw 142 to enable firing nut 146 to translate in response to rotation of firing drive screw 142. Firing nut 146 includes elongated ribs 146a disposed in spaced-apart relation to one another about an outer surface of firing nut 146. Firing nut 146 further includes an elongated lug 146b that extends along an outer surface of firing nut 146 and radially farther outward from the outer surface of firing nut 146 than elongated ribs 146a to enable firing nut 146 to translate along articulation tube 128 as elongated lug 146b of firing nut 146 slides through a slide channel 128b (FIG. 9) defined through articulation tube 128. In this regard, elongated lug 146b functions as a keel to prevent rotations as firing nut 146 rides along slide channel 128b of articulation tube 128. Firing tube 144 includes a split collar 144a on a proximal end portion thereof that interlocks with elongated ribs 146a of firing nut 146 to prevent rotation of firing nut 146 about longitudinal axis “L” when firing drive screw 142 is rotated to distally advance firing tube 144, as indicated by arrows “J” (FIG. 25). This interlocking structure of the split collar 144a and elongated ribs 146a of firing nut 146 functions as a collet and provides a low-profile design that enables firing drive screw 142 to have an increased diameter such as would be suitable for a 12 mm passage design (as opposed to the smaller diameter 8 mm passage design for which surgical stapling apparatus 10 may be utilized). A distal portion of firing tube 144 is secured to a proximal end portion of a firing rod 148 by an interlocking joint 149. The interlocking joint 149 functions to reduce overall diameter and includes a female member 144b on a distal end portion of firing tube 144 and a male member 148a on proximal end portion of firing rod 148. A distal end portion of firing rod 148 is mechanically coupled to a proximal end portion of drive beam assembly 150 via a transverse rod 148b (FIG. 18) supported on distal end portion of firing rod 148.

Drive beam assembly 150 of firing assembly 140 includes a plurality of laminates 150a, 150b, 150c having proximal hooks 151 that are secured to a distal end portion of firing rod 148. The laminates 150a, 150b, 150c have distal end portions that are secured to a drive beam 152 including a knife 152a thereon. Drive beam 152 is selectively distally advanceable through end effector 200 to drive sled 204d therethrough when firing rod 148 advances laminates 150a, 150b, 150c. When drive beam 152 distally advances, anvil and cartridge assemblies 202, 204 are configured to move from an open position to a closed position to clamp on tissue therebetween. Further distal advancement of drive beam 152 causes knife 152a of drive beam 152 to cut the clamped tissue as drive beam 152 engages and distally advances drive sled 204d through cartridge assembly 204 for causing fasteners supported therein be formed against anvil assembly 202 and secured to the clamped tissue between anvil and cartridge assemblies 202, 204. Once fired, drive beam assembly 150 can be retracted proximally away from sled 204d and proximal to spring lock 204e of cartridge assembly 204 such that spring lock 204e, which is disposed in an open or locked position (FIG. 28) after sled 204d is distally advanced, prevents further distal advancement of drive beam assembly 150 after drive beam assembly 150 is retracted to an initial position thereof.

Rotation assembly 160 of adapter assembly 100 includes third actuator 110x on a proximal end portion thereof that mechanically engages with drive mechanism 12h of housing assembly 12. Rotation assembly 160 further includes a driver 162 that is mechanically coupled to third actuator 110x and extends distally therefrom through actuator mount 113 and into inner body assembly 111. Driver 162 has a non-circular distal drive 162a to facilitate non-rotational coupling to a pinion 164 so that pinion 164 rotates with driver 162 as third actuator 110x is rotated. Pinion 164 (e.g., teeth on an outer surface thereof) is enmeshed with an annulus 166 (e.g., teeth on an inner surface thereof) having opposed mounting tabs 166a extending from diametrically opposed ends of an outer surface of annulus 166. Opposed mounting tabs 166a are mounted within tab recesses 110g defined within distal outer housing 110b. Tabs 166a of annulus 166 secure annulus 166 to upper housing 110e and to lower housing 110f of distal outer housing 110b so that distal outer housing 110b rotates relative to proximal outer housing 110a as annulus 166 is rotated by pinion 164. Rotation of distal housing 110b causes outer tube 109 and end effector 200 to rotate about longitudinal axis “L.” Rotation assembly 160 further includes a rotation ring 168 that receives annulus 166 and is supported by a proximal end portion of inner body assembly 111. Rotation ring 168 includes a stop tab 168a on an outer surface thereof that is positioned to engage a stop tab 111a depending from inner body assembly 111, whereby stop tabs 168a, 111a are positioned to function as a slip stop that enables rotation assembly 160 to rotate up to about 700 degrees. Rotation ring 168 is configured to rotate at half speed (e.g., float) such that rotation ring 168 enables two full rotations (e.g., up to about 700 degrees) minus a thickness of stop tab 168a thereof.

The various aspects disclosed herein may also be configured to work with robotic surgical systems and what is commonly referred to as “Telesurgery.” Such systems employ various robotic elements to assist the clinician and allow remote operation (or partial remote operation) of surgical instrumentation. Various robotic arms, gears, cams, pulleys, electric and mechanical motors, etc. may be employed for this purpose and may be designed with a robotic surgical system to assist the clinician during an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of clinicians may prep the patient for surgery and configure the robotic surgical system with one or more of the instruments disclosed herein while another clinician (or group of clinicians) remotely controls the instruments via the robotic surgical system. As can be appreciated, a highly skilled clinician may perform multiple operations in multiple locations without leaving his/her remote console which can be both economically advantageous and a benefit to the patient or a series of patients. For a detailed description of exemplary medical workstations and/or components thereof, reference may be made to U.S. Patent Application Publication No. 2012/0116416, and PCT Application Publication No. WO2016/025132, the entire contents of each of which are incorporated by reference herein.

Moreover, the disclosed electronic structure such as the controller(s), can include any suitable electrical components for operating the disclosed surgical stapling apparatus or components thereof. Such electrical components can include, for example, one or more controllers and/or circuitry, which may include or be coupled to one or more printed circuit boards. As used herein, the term “controller” includes “processor,” “digital processing device” and like terms, and are used to indicate a microprocessor or central processing unit (CPU). The CPU is the electronic circuitry within a computer that carries out the instructions of a computer program by performing the basic arithmetic, logical, control and input/output (I/O) operations specified by the instructions, and by way of non-limiting examples, include server computers. In some aspects, the controller includes an operating system configured to perform executable instructions. The operating system is, for example, software, including programs and data, which manages hardware of the disclosed surgical stapling apparatus and provides services for execution of applications for use with the disclosed surgical stapling apparatus. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. In some aspects, the operating system is provided by cloud computing.

In some aspects, the term “controller” may be used to indicate a device that controls the transfer of data from a computer or computing device to a peripheral or separate device and vice versa, and/or a mechanical and/or electromechanical device (e.g., a lever, knob, etc.) that mechanically operates and/or actuates a peripheral or separate device.

In aspects, the controller includes a storage and/or memory device. The storage and/or memory device is one or more physical apparatus used to store data or programs on a temporary or permanent basis. In some aspects, the controller includes volatile memory and requires power to maintain stored information. In various aspects, the controller includes non-volatile memory and retains stored information when it is not powered. In some aspects, the non-volatile memory includes flash memory. In certain aspects, the non-volatile memory includes dynamic random-access memory (DRAM). In some aspects, the non-volatile memory includes ferroelectric random-access memory (FRAM). In various aspects, the non-volatile memory includes phase-change random access memory (PRAM). In certain aspects, the controller is a storage device including, by way of non-limiting examples, CD-ROMs, DVDs, flash memory devices, magnetic disk drives, magnetic tapes drives, optical disk drives, and cloud-computing based storage. In various aspects, the storage and/or memory device is a combination of devices such as those disclosed herein.

In some aspects, the controller includes a display to send visual information to a user. In various aspects, the display is a cathode ray tube (CRT). In various aspects, the display is a liquid crystal display (LCD). In certain aspects, the display is a thin film transistor liquid crystal display (TFT-LCD). In aspects, the display is an organic light emitting diode (OLED) display. In certain aspects, on OLED display is a passive-matrix OLED (PMOLED) or active-matrix OLED (AMOLED) display. In aspects, the display is a plasma display. In certain aspects, the display is a video projector. In various aspects, the display is interactive (e.g., having a touch screen or a sensor such as a camera, a 3D sensor, a LiDAR, a radar, etc.) that can detect user interactions/gestures/responses and the like. In some aspects, the display is a combination of devices such as those disclosed herein.

The controller may include or be coupled to a server and/or a network. As used herein, the term “server” includes “computer server,” “central server,” “main server,” and like terms to indicate a computer or device on a network that manages the surgical stapling apparatus, components thereof, and/or resources thereof. As used herein, the term “network” can include any network technology including, for instance, a cellular data network, a wired network, a fiber optic network, a satellite network, and/or an IEEE 802.11a/b/g/n/ac wireless network, among others.

In various aspects, the controller can be coupled to a mesh network. As used herein, a “mesh network” is a network topology in which each node relays data for the network. All mesh nodes cooperate in the distribution of data in the network. It can be applied to both wired and wireless networks. Wireless mesh networks can be considered a type of “Wireless ad hoc” network. Thus, wireless mesh networks are closely related to Mobile ad hoc networks (MANETs). Although MANETs are not restricted to a specific mesh network topology, Wireless ad hoc networks or MANETs can take any form of network topology. Mesh networks can relay messages using either a flooding technique or a routing technique. With routing, the message is propagated along a path by hopping from node to node until it reaches its destination. To ensure that all its paths are available, the network must allow for continuous connections and must reconfigure itself around broken paths, using self-healing algorithms such as Shortest Path Bridging. Self-healing allows a routing-based network to operate when a node breaks down or when a connection becomes unreliable. As a result, the network is typically quite reliable, as there is often more than one path between a source and a destination in the network. This concept can also apply to wired networks and to software interaction. A mesh network whose nodes are all connected to each other is a fully connected network.

In some aspects, the controller may include one or more modules. As used herein, the term “module” and like terms are used to indicate a self-contained hardware component of the central server, which in turn includes software modules. In software, a module is a part of a program. Programs are composed of one or more independently developed modules that are not combined until the program is linked. A single module can contain one or several routines, or sections of programs that perform a particular task.

As used herein, the controller includes software modules for managing various aspects and functions of the disclosed surgical stapling apparatus or components thereof.

The disclosed surgical stapling apparatus may also utilize one or more controllers to receive various information and transform the received information to generate an output. The controller may include any type of computing device, computational circuit, or any type of processor or processing circuit capable of executing a series of instructions that are stored in memory. The controller may include multiple processors and/or multicore central processing units (CPUs) and may include any type of processor, such as a microprocessor, digital signal processor, microcontroller, programmable logic device (PLD), field programmable gate array (FPGA), or the like. The controller may also include a memory to store data and/or instructions that, when executed by the one or more processors, cause the one or more processors to perform one or more methods and/or algorithms.

Any of the herein described methods, programs, algorithms, or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

As can be appreciated, securement of any of the components of the disclosed apparatus can be effectuated using known securement techniques such welding, crimping, gluing, fastening, etc. Also, any of the disclosed structure can include any suitable conductive material (e.g., metallic), semi-conductive material (e.g., silicone), and/or non-conductive/insulative material (e.g., plastic).

Persons skilled in the art will understand that the structures and methods specifically described herein and illustrated in the accompanying figures are non-limiting exemplary aspects, and that the description, disclosure, and figures should be construed merely as exemplary of particular aspects. It is to be understood, therefore, that this disclosure is not limited to the precise aspects described, and that various other changes and modifications may be effectuated by one skilled in the art without departing from the scope or spirit of the disclosure. Additionally, it is envisioned that the elements and features illustrated or described in connection with one exemplary aspect may be combined with the elements and features of another without departing from the scope of this disclosure, and that such modifications and variations are also intended to be included within the scope of this disclosure. Indeed, any combination of any of the disclosed elements and features is within the scope of this disclosure. Accordingly, the subject matter of this disclosure is not to be limited by what has been particularly shown and described.

1-20. (canceled)

21. A surgical stapling apparatus, comprising:
a housing assembly; and
an adapter assembly defining a longitudinal axis and selectively attachable to the housing assembly, the adapter assembly supporting an end effector, the end effector including:
an anvil assembly;
a cartridge assembly supporting a plurality of fasteners and a sled that is movable through the cartridge assembly to fire the plurality of fasteners; and
a spring lock supported on the sled, the spring lock positioned to move from an unlocked position to a locked position when the sled moves through the cartridge assembly to prevent the cartridge assembly from being refired.

22. The surgical stapling apparatus of claim 21, further comprising a firing assembly supported in the adapter assembly, the firing assembly including a firing drive screw threadedly coupled to a firing nut.

23. The surgical stapling apparatus of claim 22, wherein the firing nut translates in response to rotation of the firing drive screw.

24. The surgical stapling apparatus of claim 23, wherein the firing nut includes elongated ribs that interlock with a split collar of a firing tube to prevent rotation of the firing nut.

25. The surgical stapling apparatus of claim 24, wherein a firing rod is secured to a distal end portion of the firing tube.

26. The surgical stapling apparatus of claim 25, wherein the firing rod is coupled to a drive beam assembly that is advanceable through the end effector.

27. The surgical stapling apparatus of claim 26, wherein the firing rod is positioned to advance the drive beam assembly through the end effector when the firing tube translates the firing rod distally.

28. The surgical stapling apparatus of claim 21, further comprising a rotation assembly having a driver coupled to a pinion, and wherein rotation of the driver causes the end effector to rotate about the longitudinal axis.

29. The surgical stapling apparatus of claim 28, wherein the pinion is coupled to an annulus and the annulus is coupled to a housing on a proximal end portion of the adapter assembly.

30. The surgical stapling apparatus of claim 29, wherein the housing is coupled to an outer tube and the outer tube is coupled to the end effector.

31. A surgical stapling apparatus, comprising:
a housing assembly;
an elongated shaft assembly defining a longitudinal axis and having a distal end portion and a proximal end portion, the proximal end portion selectively attached to the housing assembly;
an anvil assembly supported on the distal end portion of the elongated shaft assembly;
a cartridge assembly selectively attached to the anvil assembly to secure the cartridge assembly to the elongated shaft assembly, the cartridge assembly supporting a plurality of fasteners and a sled that is movable through the cartridge assembly to fire the plurality of fasteners; and
a spring lock supported on the sled, the spring lock positioned to move from an unlocked position to a locked position when the sled moves through the cartridge assembly to prevent the cartridge assembly from being refired.

32. The surgical stapling apparatus of claim 31, further comprising a firing drive screw coupled to a firing nut.

33. The surgical stapling apparatus of claim 32, wherein the firing nut translates in response to rotation of the firing drive screw.

34. The surgical stapling apparatus of claim 32, further comprising an articulation tube that is moveable to cause the anvil and cartridge assemblies to articulate relative to the longitudinal axis.

35. The surgical stapling apparatus of claim 34, wherein the firing drive screw is disposed within the articulation tube.

36. The surgical stapling apparatus of claim 31, wherein the anvil assembly includes anvil tissue stops defining cartridge tab slots therethrough, and wherein the cartridge assembly includes proximal tabs that are receivable within the cartridge tab slots of the anvil assembly to couple the cartridge assembly to the anvil assembly.

37. An adapter assembly of a surgical stapling apparatus, the adapter assembly comprising:
a proximal housing;
an elongated shaft assembly coupled to the proximal housing and defining a longitudinal axis, the elongated shaft assembly having a proximal end portion and a distal end portion; and
an end effector supported on the distal end portion of the elongated shaft assembly, the end effector including:
an anvil assembly;
a cartridge assembly selectively attached to the anvil assembly, the cartridge assembly supporting a plurality of fasteners and a sled that is movable through the cartridge assembly to fire the plurality of fasteners; and
a spring lock supported on the sled, the spring lock positioned to move from an unlocked position to a locked position when the sled moves through the cartridge assembly to prevent the cartridge assembly from being refired.

38. The adapter assembly of claim 37, further comprising a firing assembly including a firing drive screw threadedly coupled to a firing nut, the firing nut coupled to a firing tube wherein the firing tube is axially advanceable in response to axial movement of the firing nut.

39. The adapter assembly of claim 38, wherein a firing rod is secured to the firing tube, the firing rod coupled to a drive beam assembly and positioned to advance the drive beam assembly through the end effector when the firing tube translates the firing rod distally.

40. The adapter assembly of claim 37, further comprising a rotation assembly having a driver coupled to a pinion, and wherein rotation of the driver causes the end effector to rotate about the longitudinal axis.
